(19) United States
(12) Patent Application Publication
(10) Pub. No.: US 2014/0348878 A1
(43) Pub. Date: Nov. 27, 2014

(54) STRAIN OF EXOPOLYSACCHARIDE-SECRETING LACTOBACILLUS BREVIS AND APPLICATION THEREOF

(71) Applicants: Lianzhong Ai, Shanghai (CN); Benheng Guo, Shanghai (CN); Kejie Sun, Shanghai (CN); Wei Chen, Shanghai (CN); Hao Zhang, Shanghai (CN); Li Shao, Shanghai (CN); Zhengjun Wu, Shanghai (CN); Wanyi Chen, Shanghai (CN); Haibo Mu, Shanghai (CN)

(72) Inventors: Lianzhong Ai, Shanghai (CN); Benheng Guo, Shanghai (CN); Kejie Sun, Shanghai (CN); Wei Chen, Shanghai (CN); Hao Zhang, Shanghai (CN); Li Shao, Shanghai (CN); Zhengjun Wu, Shanghai (CN); Wanyi Chen, Shanghai (CN); Haibo Mu, Shanghai (CN)

(73) Assignee: BRIGHT DAIRY & FOOD CO., LTD., SHANGHAI (CN)

(21) Appl. No.: 14/363,365

(22) PCT Filed: Apr. 25, 2012
(86) PCT No.: PCT/CN2012/074639

§ 371 Date: Jun. 6, 2014

(30) Foreign Application Priority Data
Dec. 6, 2011 (CN) 201110401343.5

(51) Int. Cl. A23C 9/123; A61K 35/74; A61K 31/715; C12R 1/24

(52) U.S. Cl. CPC A23C 9/1234; C12R 1/24; A61K 35/747; A61K 31/715; A23Y 2220/13
USPC 424/282.1; 435/252.9; 424/780; 426/61; 426/43

(57) ABSTRACT

A strain of exopolysaccharide-secreting Lactobacillus brevis, BDLB0001, and an application thereof. The Lactobacillus brevis is deposited at the China General Microbiological Culture Collection Center of the China Committee of Culture Collection for Microorganisms, with a deposit number of CGMCC No. 5223. The Lactobacillus brevis secrets an elevated amount of exopolysaccharide, where the exopolysaccharide secreted is capable of eliciting B lymphocyte proliferation to enhance immunity, and has application prospects in medicaments, healthcare products and food products for immunity enhancement.

### FIELD OF INVENTION

The present invention relates to the field of microbial technology. More specifically, the invention relates to a strain of exopolysaccharide-secreting Lactobacillus Brevis and application in foods thereof

### PRIOR ARTS

Lactic acid bacteria (LAB) is a generic term which refers to a kind of bacteria that can use fermentable sugar to produce a large amount of lactic acid. It has been found that this kind of bacteria includes at least 23 genera in taxonomy in nature at present. In the fields of food and medicine, the widely applied Lactic acid bacteria mainly include Lactobacillus, Streptococcus, Enterococcus, Lactococcus, Pediococcus and Leuconostoc, etc. Lactic acid bacteria are the main source of probiotics. Many Lactic acid bacteria, which are inherent in human gut, have already been proved to have important physiological activities, such as improving the human intestinal flora, regulating the body's immunity, suppressing tumors, reducing serum cholesterol and regulating blood pressure, etc.

Significant progress has been made on the application of Lactic acid bacteria in fermented dairy products and microecologics. Current research is focused on how to develop new microbial starter cultures which can endow specific functional properties to fermented milk. The mechanism that the known Lactic acid bacteria display their functions include colonization, improving the intestinal environment by main metabolites such as lactic acid, besides these, some secondary metabolites, such as bacteriocin and exopolysaccharides etc., also play very important roles. Among these metabolites, exopolysaccharides of Lactic acid bacteria (LAB EPS) which have theoretical and practical application value have aroused the interest of many scholars at home and abroad.

LAB EPS is a kind of polysaccharide produced and secreted into the extracellular space by Lactic acid bacteria. Compared with other microbial polysaccharides, LAB EPS has its own characteristics, such as good rheological properties, the ability of improving flavor, texture and nature of fermented milk. Thus, the LAB EPS is a safe food additive which could thicken, stabilize, emulsify, moisturize the products and form gel thereon, and then make products be more delicate and uniform in texture and taste lubricant. EPS also has good physiological functions, such as enhancing mucosal absorption, anti-tumor, anti-ulcer, immune regulation, lowering cholesterol and lowering blood pressure, etc. Therefore, the study on LAB which could produce extracellular polysaccharide is of great significance and economic value to improve dairy production and develop fermented dairy products that bear specific functional properties. To exploit LAB EPS bearing probiotic functions is becoming a hot spot of current researches.

### Content of the Present Invention

The technical problem to be solved in the present invention is for overcoming the shortcomings that there is no Lactic acid bacteria produces high content of exopolysaccharides in prior art to provide a high-productive strain of exopolysaccharide-secreting Lactobacillus brevis.

The technical solutions of the present invention are as follows.

The first technical solution of the present invention is a strain of exopolysaccharide-secreting Lactobacillus brevis is deposited in China General Microbiological Culture Collection Center (CGMCC) with an accession number of CGMCC No. 5223.

The second technical solution of the present invention is a bulk starter culture of Lactobacillus brevis CGMCC No. 5223 which is prepared according to a method comprising the following step (a) or (b):

(a) inoculating the strain of Lactobacillus brevis CGMCC No. 5223 into sterilized milk and culturing till the milk begin to curd, continuing the activated culture for two generations to obtain the mother starter culture; inoculating said mother starter culture with a ratio of 3-5% (v/v) into new sterilized milk and culturing till the new sterilized milk begins to curd, and then obtaining said bulk starter culture;

(b) inoculating the strain of Lactobacillus brevis CGMCC No. 5223 into liquid medium to culture for activation, continuing the activated culture for two generations, inoculating the activated culture obtained with a ratio of 2-4% (v/v) into new liquid medium and culturing for 16-18 h, obtaining the cell sediment through solid-liquid separation, and suspending the cell sediment in sterilized milk, and then obtaining said bulk starter culture.

In procedure (a), the method of cultureing till the milk begin to curd is a conventional method in the art, and preferably refers to culture at the temperature of 37° C. for 14-16 h.

In procedure (b), the method of cultureing for activation in liquid medium is a conventional method to activate Lactobacillus brevis in the art, and preferably refers to culture at the temperature of 37° C. for 12-16 h. Said liquid medium is conventional liquid medium for culturing Lactobacillus brevis in the art, and preferably refers to MRS liquid medium. The method of solid-liquid separation is a conventional solid-liquid separation method to separate thalli of fermentation liquor in the art including centrifugation and filtration, etc.; preferably the method of solid-liquid separation in the present invention refers to centrifugation, and more preferably refers to centrifugation at the temperature of 4° C. for 15 min with the speed of 4000 r/min.

Preferably, a viable count of bacteria in said bulk starter culture of Lactobacillus brevis CGMCC No. 5223 in the present invention is no less than 10⁹ cfu/mL.

The third technical solution of the present invention is use of Lactobacillus brevis CGMCC No. 5223 in fermented foods.

Therein, said fermented foods refer to conventional fermented foods, preferably refer to Lactic acid bacteria-containing milk beverage or fermented milk.

Preferably, said Lactic acid bacteria-containing milk beverage is prepared according to a method comprising the following steps: cooling raw milk after sterilization, mixing with said bulk starter culture of Lactobacillus brevis CGMCC No. 5223 uniformly to make the concentration of Lactobacillus brevis CGMCC No. 5223 up to 10⁶ cfu/mL or more, and then obtaining said Lactic acid bacteria-containing milk beverage

Therein, the method of said sterilization refers to conventional methods for raw milk sterilization, preferably sterilization with UHT, more preferably pasteurization at the temperature of 95° C. for 20 min or high temperature sterilization at the temperature of 140° C. for 2 s. Said bulk starter culture of Lactobacillus brevis CGMCC No. 5223 is added after cooling the sterilized milk, therein, the cooling temperatureis conventional, preferably is 40° C.

Preferably, said fermented milk is prepared according to the method comprising the following steps: cooling raw milk after sterilization, inoculating a ratio of 3-5% (v/v) of the bulk starter culture of Lactobacillus brevis CGMCC No. 5223 as well as a ratio of 3-5% (v/v) of mutualistic commercial starter cultures into the sterilized milk, mixing uniformly and fermenting till the titration acidity reaches to 0.6-0.7 calculated by lactic acid, and then obtaining the fermented milk containing said Lactobacillus brevis.

Therein, the method of sterilization refers to conventional methods for raw milk sterilization, preferably sterilization with UHT, more preferably pasteurization at the temperature of 95° C. for 20 min or high temperature sterilization at the temperature of 140° C. for 2 s. Said bulk starter culture of Lactobacillus brevis CGMCC No. 5223 is added after cooling the sterilized milk, therein, the cooling temperature is conventional, preferably is 37° C. The temperature for fermentation is conventional, preferably 37° C. The mutualistic commercial starter cultures refer to conventional commercial starter cultures, such as Lactobacillus Bulgaria.

The forth technical solution of the present invention is the exopolysaccharides extracted from strain of Lactobacillus brevis CGMCC No. 5223.

Therein, the method of said extraction refers to conventional methods for extracting microbial exopolysaccharides, preferably refers to a method comprising the following steps: centrifuging the fermentation liquor of Lactobacillus brevis CGMCC No. 5223 after boiling in order to remove thalli and condensed protein, precipitating protein from supernatant via trichloroacetic acid method to remove the protein, obtaining a precipitate by adding alcohol, dissolving the precipitate obtained in water and then dialyzing with water.

Therein, the fermentation liquor of Lactobacillus brevis refers to conventional fermentation liquor of Lactobacillus brevis, preferably refers to the fermentation liquor that is obtained by the fermentation comprising the steps of inoculating 5% (v/v) Lactobacillus brevis into 12% (w/w) skimmed milk containing 1% (w/v) glucose and ferment; preferably refers to the fermentation liquor that is obtained by the fermentation at 30° C. for 30 h.

Therein, the preferred condition of centrifugation is 10000 g at 4° C. for 20 min. Trichloroacetic acid method is a conventional method of removing protein. Preferably, trichloroacetic acid is added to a final concentration of 4% (w/v) and stand overnight, then centrifugate with the speed of 10000 g at 4° C. for 20 min to remove the precipitated proteins. The method of precipitating with alcohol is also a conventional method, preferably with ethanol, i.e., add ethanol to a final concentration of 75% (v/v) and stand at 4° C. for 24 h, then centrifuge with the speed of 10000 g at 4° C. for 20 min to obtain the precipitate.

The fifth technical solution of the present invention is use of the exopolysaccharides extracted from strain of Lactobacillus brevis CGMCC No. 5223 for use in immune-enhancing medicament, healthcare product or food product.

Unless otherwise indicated, all materials or reagents used in the present invention are commercially available.

Compared to the prior art, the advantageous effects of the present invention is as follows: the present invention provides a strain of Lactobacillus brevis CGMCC No. 5223 which secrets an elevated amount of exopolysaccharide, where the exopolysaccharide secreted is capable of eliciting B lymphocyte proliferation to enhance immunity, and has broad application prospects in medicaments, healthcare products and food products for immunity enhancement.

### Deposit Information of the Biological Material Sample

The strain of Lactobacillus brevis CGMCC No. 5223 provided by the present invention is deposited in China General Microbiological Culture Collection Center (CGMCC) which is located in No. 1 Yard, West Beichen Road, Chaoyang District, Beijing with the postal code 100101 on Sep. 6, 2011. Accession number of the strain is CGMCC No. 5223.

### BRIEF DESCRIPTION OF THE DRAWINGS

Combined with the description of figures, the feature and the advantageous effects of the present invention are illustrated as follows.

FIG. 1 is the colony morphology of Lactobacillus brevis CGMCC No. 5223.

FIG. 2 is the cellular morphology of Lactobacillus brevis CGMCC No. 5223 (×1000).

FIG. 3 is the growth curves of Lactobacillus brevis CGMCC No. 5223.

FIG. 4 is the optimum growth temperature of Lactobacillus brevis CGMCC No. 5223.

FIG. 5 is the optimum pH of Lactobacillus brevis CGMCC No. 5223.

### DETAILED DESCRIPTION OF THE PREFERRED EMBODIMENT

The present invention collected samples from habitats of Lactic acid bacteria and screened wild strains of Lactic acid bacteria which produced exopolysaccharides, and then study the biological activity of said exopolysaccharides and exploit new probiotics.

The present invention provides a Lactobacillus brevis strain BDLB0001.

The present invention isolated a Lactic acid bacteria strain BDLB0001 from naturally fermented pickles. By use of microbial characteristics like morphological characteristics, culture characteristics, physiological and biochemical traits and genetic characteristics 16s rDNA, the Lactic acid bacteria strain BDLB0001 was identified as Lactobacillus brevis, which is deposited in China General Microbiological Culture Collection Center (CGMCC) with accession number of CGMCC No. 5223 on Sep. 6, 2011.

The morphological characteristics of said Lactobacillus brevis CGMCC No. 5223 in the present invention are described as below:

Colony characteristics: the strain grows well after streaked on MRS agar plate and then incubated anaerobically at 37° C. for 48 h, and the colony morphology is shown in FIG. 1. The colonies are 0.2-2 mm in size, rounded with neat edge, front slightly raised, milky white and mixed up with little yellowish color, non-transparent, with humid and smooth surface, and could be brushed if be picked.

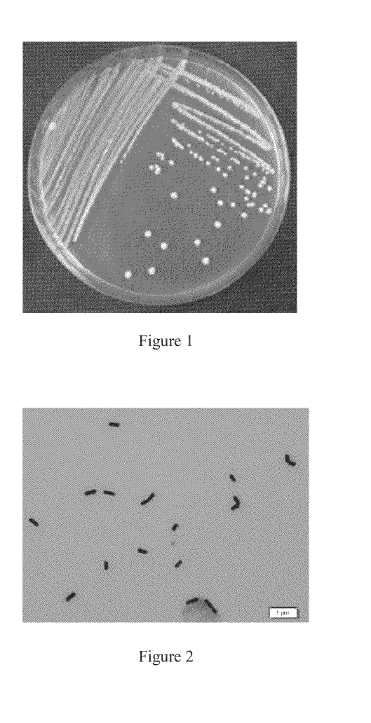

Strain characteristics: the bacterium is short rod-shaped with rounded terminals (see FIG. 2), and the strain exist in form of single cells or pairs with uniform color. Being gram positive, the bacteria do not generate spores with general size of 0.9 μm×1.8 nm.

The culture characteristics of said Lactobacillus brevis CGMCC No. 5223 in the present invention are described as below:

The minimum growth temperature of Lactobacillus brevis strain BDLB0001 is 15° C. while the maximum growth temperature is 40° C., and the optimum growth temperature is between 30° C. and 40° C. The highest initial pH for growth is 8.0 and the lowest initial pH for growth is 4.0, and the optimum initial pH for growth is 6.0. Said Lactobacillus brevis strain BDLB0001 has a relatively short lag phase and grows into the logarithmic phage at the time of 2 h and the stationary phase at the time of 10 h. The strain of Lactobacillus brevis BDLB0001 has a good bile salt tolerance and could grow well with the bile salt concentration ranging from 0.1% to 0.4%; and also the strain BDLB0001 grows well when the concentration of NaCl is not more than 7% as the strain could tolerate 9% NaCl.

Said Lactobacillus brevis strain BDLB0001 in the present invention is derived from traditional fermented food and belongs to the Generally Recognized As Safe (GRAS) strains, so it can be used in Lactic acid bacteria-containing foods.

Therefore, the present invention also relates to an application of said Lactobacillus brevis BDLB0001 in fermented foods.

The present invention also provides a bulk starter culture of said Lactobacillus brevis BDLB0001.

Preferably, the bulk starter culture in the present invention is prepared according to the following preparation method: Lactobacillus brevis strain BDLB0001 is inoculated into sterilized 12% (w/v) skimmed milk to culture at the temperature of 37° C. for 14-16 h till the milk begin to curd, the activated culture is continued for two generations to obtain a mother starter culture; then said mother starter culture is inoculated into new sterilized 12% (w/v) skimmed milk with a ratio of 3-5% (v/v) to culture at the temperature of 37° C. for 14-16 h till the new skimmed milk begin to curd, then said bulk starter culture is obtained with a viable bacteria count of about 10⁹ cfu/mL in the curdled milk. Alternatively, Lactobacillus brevis strain BDLB0001 is inoculated into MRS liquid medium to culture for activation at the temperature of 37° C. for 12-16 h, and the activated culture is continued for two generations, then the activated culture obtained is inoculated into new MRS liquid medium with a ratio of 2-4% (v/v) to culture for 16-18 h, and then the cell sediment is obtained via centrifugation at the temperature of 4° C. for 15 min with the speed of 4000 r/min, and the cell sediment is suspended in a certain amount of sterilized skimmed milk, then said bulk starter culture is obtained.

Preferably, said Lactic acid bacteria-containing milk beverage in the present invention is prepared by the following procedure: Raw milk is cooled to 40° C. after pasteurization at the temperature of 95° C. for 20 min or high temperature sterilization at the temperature of 140° C. for 2 s, and said bulk starter culture of Lactobacillus brevis strain BDLB0001 is added to make the concentration of the strain up to more than 10⁶ cfu/mL, then the product is kept in cold storage at 4° C., i.e., said Lactic acid bacteria-containing milk beverage is obtained.

Preferably, said fermented milk in the present invention is prepared by the following procedure: Raw milk is sterilized via pasteurization at the temperature of 95° C. for 20 min or high temperature sterilization at the temperature of 140° C. for 2 s and cooled to 37° C., then a ratio of 3-5% (v/v) of the bulk starter culture of Lactobacillus brevis strain BDLB0001 is added, and then a ratio of 3-5% (v/v) of mutualistic commercial starter cultures which are used for preparing fermented milk is added into the sterilized milk, and after being mixed uniformly, the inoculated milk is fermented till the titration acidity reaches to 0.6-0.7 calculated by lactic acid, then cool the product to 40° C. and kept it in cold storage, i.e., the fermented milk containing said Lactobacillus brevis is obtained.

The following embodiments further illustrate the invention, but the present invention is not limited thereto. Unless otherwise indicated detailedly, the experimental methods of the present invention are usually in accordance with conventional conditions, or in accordance with the conditions recommended by the manufacturer. Said “room temperature” in the embodiments refers to the temperature of the operating room used for experiments, generally is 25° C.

### Embodiment 1

### Collection and Isolation of Lactobacillus brevis Strain BDLB0001

(1) Sample collection

Collect samples from naturally fermented pickles, traditional fermented dairy products (yogurt, sour horse milk, etc.), raw milk, raw dough, fermented sausages, salami, kefir grains (Tibetan kefir), silage and baby feces, etc. The collected samples are kept storing in ice box and brought back to the laboratory at low temperatures, and then placed in a refrigerator at 4° C., then separate the Lactic acid bacteria as early as possible.

(2) Sample pretreatment

Put 20 g solid sample (or 20 mL liquid sample) into 250 mL flask (with glass beads) containing 180 mL sterilized 0.1% peptone water (peptone 1 g, distilled water 1000 g), and stand 20 min after shaking for reserve.

(3) Preliminary isolation of exopolysaccharide-secreting strains

Serial dilutions of the samples are carried out using sterilized 0.1% peptone water with a ratio of 1:10 in volume, and in each dilution, 0.1 mL diluted sample is coated on to MRS agar plate, M17 agar plate, SM agar plate, Modified MRS agar plate and ESM plate, respectively. The plates are placed for anaerobic culture at a constant temperature of 37° C. for 24-48 h, and then pick single colonies which is sticky and presents stringy state obviously by sterilized toothpicks. Then the single colony are streaked onto corresponding agar plates to obtain purified single colonies for Gram stain and catalase test. And the purified strains obtained are deposited in corresponding isolation mediums supplemented with 20% glycerol as protective agent and cryo-preserved at −20° C. for cryopreservation. Therein, the medium formulations are as follows:

SM medium (g/L): 120 g skimmed milk powder, 10 g glucose, 880 g water.

ESM medium (g/L): 90 g skimmed milk powder, 3.5 g yeast extract, 3.5 g peptone, 20 g glucose (Van den Berg, D. J. C., A. Smits, B. Pot, A. M. Ledeboer, K. Kersters, J. M. A. Verbakel, and C. T. Verrips. 1993. Isolation, screening and identification of Lactic acid bacteria from traditional food process and culture collections. Food Biotechnol. 7:189-205).

MRS medium (selective medium for Lactobacillus, Merck, Germany)

M17 medium (medium for Lactococcus, BD Difco)

Modified MRS medium: the glucose content of the MRS medium is changed into 50 g/L and other components remain unchanged.

700 strains are isolated from different samples on MRS agar plate, M17 agar plate, Modified MRS agar plate, ESM plate and SM agar plate. These strains present stringy, sticky and mucoid states on the isolation plates.

(4) Exopolysaccharide-secreting strains

The isolated strain obtained from the plate is inoculated into MRS liquid culture medium and culture for 18 h, then 1% (V/V) of the inoculum is inoculated into MRS liquid medium containing 50 g/L glucose and ferment at 30° C. for 24 h. Take 20 mL of the culture medium obtained for boiling water bath for 10 min, then after cooling to the room temperature, the supernatant is added trichloroacetic acid with the mass fraction of 80% to a final concentration of 4% (m/v). Stand overnight at 4° C. and centrifuge with the speed of 10000 g for 20 min, then slightly pour the supernatant into dialysis bag with the MWCO (Molecular Weight Cut Off) of 14000 for dialysis using deionized water for 72 h. Change the water once every 8 h in the process of dialysis and keep in constant volume finally. The content of exopolysaccharide is measured by sulfuric acid-phenol method (Dubois M, Gilles K A, Hamilton J K, Pebers P A, Smith F. (1956). Colorimetric method of determination of sugars and related substances. Analytical chemistry. 28(3):350-356). The experimental results are shown in Table 1. The strain 22-22, as can be seen from Table 1, produces relatively high content of crude polysaccharides and is selected and named as BDLB0001.

TABLE 1
Preliminary isolation of exopolysaccharide-secreting Lactic acid bacteria
strains (culture at 30° C. for 24 h)

| strain number | EPS (mg/L) |
|---|---|
| 4-21 | 60.98 |
| 5-28 | 81.6 |
| 9-9 | 131.96 |
| 17-5 | 109.03 |
| 17-15 | 135.79 |
| 17-16 | 126.79 |
| 18-9 | 125.48 |
| 19-16 | 117.61 |
| 19-19 | 115.87 |
| 26-9 | 81.53 |
| 26-8 | 158.91 |
| 22-22 | 161.88 |
| 27-2 | 93.12 |
| 33-4 | 107.79 |
| 33-10 | 100.23 |

### Embodiment 2

### Identification of Lactobacillus brevis Strain BDLB0001

(1) Physiological and biochemical tests

The BDLB0001 strain, a kind of bacillus, which is Gram-positive, peroxidase-negative and with no bacterial motility, can grow at the temperature of 15° C. and 40° C. The strain does not hydrolyze starch and liquefy gelatin, and neither produce hydrogen sulfide. Acid is produced and no gas is produced when the strain is fermented with glucose. The strain is negative in benzidine test and indole test, and positive in methyl red test.

(2) Identify the strain by sugar fermentation tests

Pick up a small amount of culture medium of strain BDLB0001 to streak onto the MRS solid plate for anaerobic culture at 37° C. for 24-48 h. Single colony picked from the plate is inoculated into API 50 CHL liquid medium (bioMérieux China Ltd., API 50 CHL Medium) to prepare bacterial suspension, then the bacterial suspension is placed at 37° C. for anaerobic culture for 24-48 h after adding API 50 CHL identification reagent strips (bioMérieux China Ltd.). The fermentation results of 49 kinds of carbohydrates of the strain are recorded and input into the Merieux authentication software API LAB PLUS, and results are shown in Table 2. Via database queries, the BDLB0001 strain in the present invention shows a homology of 99.8% with the Lactobacillus brevis, thus the BDLB0001 strain in the present invention is identified as Lactobacillus brevis preliminarily.

TABLE 2
Carbon source utilization of the BDLB0001 strain

| carbohydrate | result | carbohydrate | result |
|---|---|---|---|
| Glycerin | − | Salicin | − |
| Erythritol | − | D-cellobiose | − |
| D-arabinose | − | D-maltose | + |
| L-arabinose | + | D-lactose | − |
| D-ribose | + | D-melibiose | − |
| D-xylose | + | D-sucrose | − |
| L-xylose | − | D-trehalose | − |
| D-adonitol | − | Inulin | − |
| Methyl-β-D-xylopyranoside | − | D-melezitose | − |
| D-galactose | −+ | D-raffinose | − |
| D-glucose | + | Starch | − |
| D-fructose | −+ | Glycogen | − |
| D-mannose | − | Xylitol | − |
| L-sorbose | − | D-gentiobiose | − |
| L-rhamnose | − | D-Toulon sugar | − |
| Dulcitol | − | D-lyxose | − |
| Inositol | − | D-tagatose | − |
| Mannitol | − | D-fucose | − |
| Sorbitol | − | L-fucose | − |
| Methyl-α-D-mannopyranoside | − | D-arabitol | − |
| Methyl-α-D-glucopyranoside | − | L-arabitol | − |
| N-acetylglucosamine | + | Potassium | + |
| | | gluconate | |
| Laetrile | − | 2-keto-potassium | − |
| | | gluconate | |
| ARBULIN | − | 5-keto-potassium | + |
| | | gluconate | |
| Esculin ferric citrate | + | | − |

Note:
“+” means positive reaction.
“−” means negative reaction.
“−+” means the carbon source utilization cannot be determined.

(3) 16s rDNA sequence analysis of strain BDLB0001

Genomic DNA extraction method of strain BDLB0001: single colony of purified BDLB0001 is picked and inoculated into 1 mL MRS liquid medium, and after cultured at 37° C. for 14 h, centrifugate (5000 g, 10 min) the bacterial liquid to collect the thalli cells. Extract the genomic DNA by using the genomic DNA extraction kit (TIAN GEN company). PCR amplification is carried out by using two synthetic universal primers (16s 27F: GAGAGTTTGATCCTGGCTCAG; 16s 1492R: CGGCTACCTTGTTACG ACTT). The PCR products are recovered by using extraction kit (BioFlux) after cutting gel, then purifies the recovered PCR products for sequencing (Invitrogen biotechnology companies). The 16s rDNA nucleotide sequence of the strain BDLB0001 obtained is 1442 bp in length (shown as SEQ ID NO: 1 in the sequence listing) and the sequence is input into GenBank for Blast analysis with accession number of JN86880. The strain BDLB0001 has the highest homology of 99% with L. brevis ATCC 14687 (GenBank accetion number: EF 120367).

According to the argument of Goodfellow and O'Donnell, for the DNA, if G+C (mol %) ≦10%˜12%, and meanwhile for the 16S rRNA, if the sequence homology ≧95%, the strains can be classified as a genus. And according to the argument of Embley and Stackebrangdt, for the 16S rRNA, if the sequence homology ≧97%, the strains can be classified as a species. It can be inferred: the L. brevis ATCC 14687 strain and the BDLB0001 strain belong to the same species. According to these arguments, the strain BDLB0001 is identified as Lactobacillus brevis.

Based on microbial characteristics like morphological characteristics, physiological-biochemical characteristics and genetic characteristics, i.e., 16s rDNA, strain of Lactic acid bacteria BDLB0001 is identified as Lactobacillus brevis. The strain was deposited in China General Microbiological Culture Collection Center (CGMCC) with accession number of CGMCC No. 5223.

### Embodiment 3

### Growth Characteristics of the BDLB0001 Strain

(1) Drawing the growth curve of the BDLB0001 strain

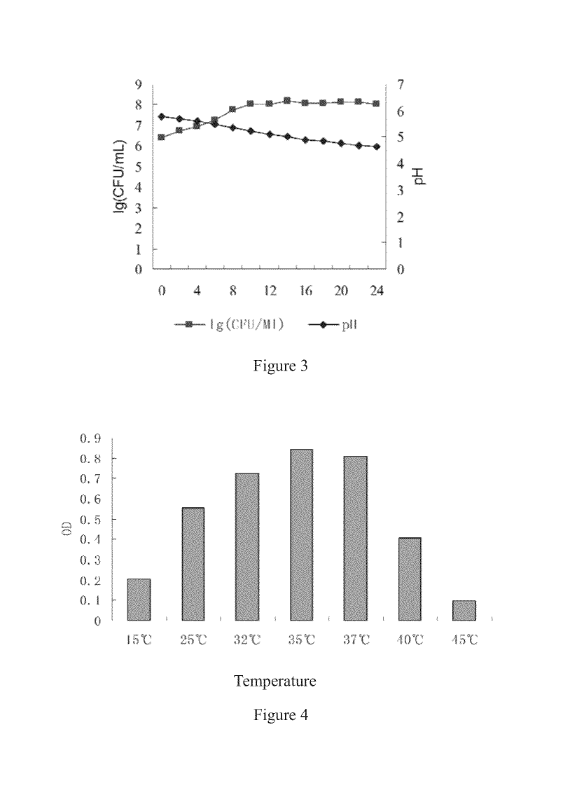

The activated strain of Lactobacillus brevis BDLB0001 is inoculated into MRS liquid medium with a ratio of 1% (VAT) and cultured at 37° C. for 24 h. And at 600 nm, the viable bacteria count and pH value of the culture medium is measured. The pH value of the culture medium is measured with a pH meter, while the viable bacteria count is measured by using the plate count method. The logarithm of the viable bacteria count and pH value are plotted against time, and the growth curve of the strain BDLB0001 in MRS liquid medium is obtained. The results (FIG. 3) showed that: The strain of Lactobacillus brevis BDLB0001 grows rapidly in MRS liquid medium, and it grows into the logarithmic phage at the time of 2 h and the stationary phase at the time of 10 h. With prolonged incubation time, the pH lower continuously as the strain begins to produce acid and the degree of pH decline slows after entering the stable stage. At the end of 24 h's culture, the pH value of the culture medium is 4.63 and the viable bacteria count in the culture medium can reach 10⁸ CFU/mL.

(2) Measuring the optimum growth temperature of the BDLB0001 strain

The activated Lactobacillus brevis BDLB0001 strain is inoculated into 10 mL MRS liquid medium with a ratio of 1% (V/V) and cultured at 15° C., 37° C., 40° C., 45° C. and 65° C. for 8 h, with the uninoculated MRS liquid medium as a control, then the OD values of the culture medium at different culture temperatures are measured at 620 nm, and the optimum growth temperature is determined according to the OD values. The results shows (FIG. 4) that the Lactobacillus brevis BDLB0001 strain has a wide temperature range for growth from 15° C. to 40° C. and can grow well at the growth temperature between 30° C. and 40° C., and the optimum growth temperature is 35° C.

(3) Measuring the optimum growth pH of the BDLB0001 strain

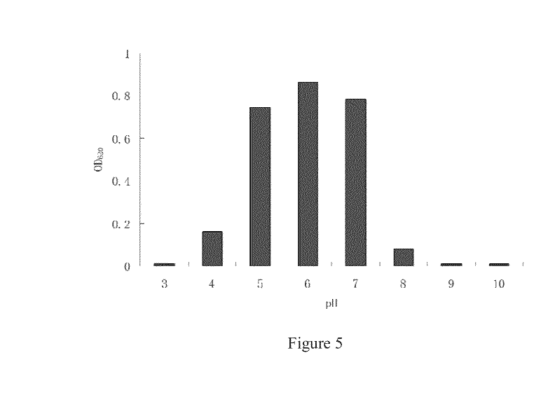

The activated Lactobacillus brevis BDLB0001 strain is inoculated into MRS liquid medium with different initial pH of 3.0, 4.0, 5.0, 6.0, 7.0, 8.0, 9.0 and 10.0, respectively and then cultured at 37° C. for 16 h, and the uninoculated MRS liquid medium with the same initial pH is as a control, then the OD values of the culture medium are measured at 620 nm, and the optimum growth pH is determined according to the OD values. The results shows (FIG. 5) that the Lactobacillus brevis BDLB0001 strain grows well with the initial pH of 4.0-7.0 and the optimum pH is determined to be 6.0.

(4) Bile tolerance test of the Lactobacillus brevis BDLB0001 strain

The activated Lactobacillus brevis BDLB0001 strain is inoculated into MRS liquid medium with different concentrations of sodium taurocholate (TCA) (with mass fraction of 0.0%, 0.05%, 0.1%, 0.15%, 0.2%, 0.25%, 0.3%, 0.35% and 0.4%, respectively), and then cultured at 37° C. for 24 h, then the OD values of the culture medium are measured at 620 nm, and determined the bile tolerance according to the OD values. The bile salt content in human intestinal is between 0.03%-0.3% with fluctuations, and strains which could grow and metabolite in normal physiological concentration of bile salts in the intestinal transit was likely to survive in the process of intestinal transit. As shown in Table 3, along with the increasing concentration of bile salts, the OD value decrease and the strains exhibit no adaptation. The Lactobacillus brevis BDLB0001 strain exhibits good tolerance to bile salts as it can grow well with the bile salt concentration ranging from 0.1% to 0.4%. The results suggest that the Lactobacillus brevis BDLB0001 strain can survive, grow normally and reproduce in the small intestine and there is potential for the development of being probiotics.

TABLE 3
The growth conditions of Lactobacillus brevis BDLB0001
strain in the medium with different concentrations of bile salt

| | Mass fraction of TCA (%) | | | | | | |
|---|---|---|---|---|---|---|---|
| | 0 | 0.1 | 0.15 | 0.2 | 0.25 | 0.3 | 0.4 |
| OD₆₂₀ | 2.4825 | 2.4549 | 2.4159 | 1.3704 | 0.5868 | 0.5156 | 0.492 |

(5) NaCl tolerance test of the Lactobacillus brevis BDLB 0001 strain

The activated Lactobacillus brevis BDLB0001 strain is inoculated into MRS liquid medium with different concentrations of NaCl (with mass fraction of 0%, 2%, 4%, 6%, 7%, 8%, 9%, 10% and 11%, respectively), and then cultured at 37° C., then with bromcresol purple as an indicator, the NaCl tolerance is observed. The results are shown in Table 4. The Lactobacillus brevis BDLB 0001 strain grows well when the growth medium contains no more than 7% NaCl, grows slowly when the growth medium contains 9% NaCl, and no growth can be observed when the growth medium contains more than 10% NaCl. The results suggest the BDLB 0001 strain has good NaCl tolerance.

TABLE 4
NaCl tolerance of the Lactobacillus brevis BDLB 0001 strain

| Concentrations of NaCl | |
|---|---|
| (%) | Growth condition |
| 0 | ++ |
| 2 | ++ |
| 4 | ++ |
| 6 | ++ |
| 7 | ++ |
| 8 | + |
| 9 | + |
| 10 | − |
| 11 | − |

Notes:
“++” means grows well,
“+” means growth can be observed,
“−” means no growth can be observed

### Embodiment 4

### Extraction of the Extracellular Polysaccharides Produced by Lactobacillus brevis BDLB0001

(1) Strain activation: The strain of Lactobacillus brevis BDLB0001 is inoculated into MRS liquid and cultured at 37° C. for 12-16 h for activation, the activated culture is continued for two generations.

(2) Seed culture: The activated strain of Lactobacillus brevis BDLB0001 is inoculated into 12% (w/w) skimmed milk containing 1% (w/v) glucose which is sterilized at 115° C. for 15 min, and cultured at 37° C. for 14-16 h till the milk begin to curd, the activated culture is continued for two generations, and the products obtained is used as a mother starter culture.

(3) Fermentation: The strain of Lactobacillus brevis BDLB0001 is inoculated into 12% (w/w) skimmed milk containing 1% (w/v) glucose with a ratio of 5% (v/v) and cultured at 30° C. for 30 h.

(4) Extraction and Purification of EPS: Take the fermentation liquor prepared above for boiling water bath for 10 min first to inactivate the enzyme which could degrade polysaccharides, then centrifuge (20 min, 10000 g, 4° C.) to remove the thalli cells and the coagulated proteins. The supernatant is concentrated to ½ of the original volume, and add trichloroacetic acid to a final concentration of 4% (w/v), stand overnight, and then centrifugate (20 min, 10000 g, 4° C.) to remove the precipitated proteins. 95% (v/v) ethanol is added into the concentrated solution to a final concentration of 75% (v/v), stand at 4° C. for 24 h, then centrifugate (20 min, 10000 g, 4° C.). The precipitate obtained is dissolved in deionized water, centrifugation (20 min, 10000 g, 4° C.) to remove the precipitate, and the supernatant is dialyzed in deionized water for 72 h. Change the water once every 8 h in the process of dialysis and via freeze dry the crude polysaccharide sample is obtained.

### Embodiment 5

### Immune Activity of the Polysaccharides In Vitro

Cytotoxicity assay and the proliferative response of T/B lymphocytes of EPS

Aseptically removed BALB/C mouse spleen and make into spleen cell suspension. Lymphocytes are isolated by lymphocyte separation medium (Shanghai SABC) and washed twice with PBS buffer solution (containing 0.144 g KH₂PO₄, 9.0 g NaCl, 0.795 g Na₂HPO₄ 7H₂O per liter, pH=7.4), then adjust the cell concentration to 1×10⁶/mL to obtain a splenic lymphocytes suspension with RPMI1640 medium (Biosharp Amresco Company). 150 μL splenic lymphocytes suspension and 50 μL polysaccharide samples of different concentrations (10 μg/mL, 100 μg/mL, 1000 μg/mL) are added to a 96-well culture plate. Elicit T lymphocyte proliferation with mitogen concanavalin A (ConA, 5 μg/mL, Sigma) and elicit B lymphocyte proliferation with lipopolysaccharide (LPS, 10 μg/mL). Negative control group (containing only splenic lymphocytes suspension) and positive control group (add mitogen) are also set up. In cytotoxicity assays, do not add mitogen. Three repeats are set up for each experimental group and then cultured at 37° C. for 72 h with 5% CO₂ and saturated humidity.

(1) Cytotoxicity test: Use MTT assay (Xu De-Yi, Jia Hong-Bin “5-HT3 receptors in amygdala mediate neuroimmunomomodulation in rats” [J] Acta Physiologica Sinica, 2001, 53 (5): 349-354) to take the test. 4 h before the end of culturing, each well is added 20 μL MTT (5 g/L, Sigma) and continued to culture for 4 h. After culturing, 150 μL DMSO is added in. Then A₅₇₀ values is measured at 570 nm via the ELISA apparatus. Therein, the MTT solution is prepared by the following process: MTT is dissolved with D-hank's solution. And stir to dissolve completely, then keep in constant volume to make the concentration of MTT be 5 mg/mL.

MTT assay develops rapidly and is widely applied because of its short experimental period, simple operation, high sensitivity and good reproducibility, and it is very important in the fields of cell biology, radiation biology and immunology. The principle of MTT assay is that the succinate dehydrogenase of mitochondria in the living cells can make the yellow MTT restore to poorly soluble blue-violet product and deposite the product in cells (dead cells have no such function). After dissolution with dimethyl sulfoxide (DMSO), the absorbance at a certain wavelength, which is positively correlated with the metabolic capacity of the living cells and reflects the proliferation of cells, is measured via the ELISA apparatus.

Through the MTT assay, it is found that there is no significant differences of the OD values between the in vitro culture medium of mouse spleen lymphocyte with different concentrations of crude exopolysaccharides and the control group, the results are shown in Table 5. The results suggest that the crude exopolysaccharides displays no cytotoxicity.

TABLE 5
Cytotoxicity test of the crude exopolysaccharides

| | Concentration | | | cyto- |
|---|---|---|---|---|
| | μg/mL | OD value | P value | toxicity |
| control | — | 0.124 ± 0.001 | | |
| crude | 10 | 0.137 ± 0.005 | 0.0329 | No |
| exopolysaccharides | 100 | 0.127 ± 0.037 | 0.0034 | No |
| | 1000 | 0.202 ± 0.009 | 0.2614 | No |

(2) Cell proliferation test: Use ³H-TdR incorporation method (Guo Qu-lian, Zhang Yang-de, Zou Wang-yuan, et al. “Effects of fintrathecal morphineonspleen T-lymphocyte proliferation and NK cell activity in rats”, [J] Chinese Journal of Anesthesiology, 2005, 25 (2): 118-121). 8 h after cultivation, each well is added in 20 μL 3H-TdR (370 kBq/mL). After cultivation, cells were harvested onto the 49 glass-fiber filter paper, and the paper is dried and placed in the PPO—POPOP (Sigma) scintillation solution overnight, and measure CPM value of each tube by the liquid scintillation spectrometer.

Therein, the ³H-TdR working solution is prepared by the following procedure: the stock solution is 37 MBq/mL, the specific strength of radioactivity is 0.925 TBq/mmol Dilute to the final concentration of 370 kBq/mL with RPMI 1640 culture medium before use.

The scintillation solution is prepared by the following procedure: After being added in a small amount of xylene, the POPOP (0.1-0.3 g) is dissolved in water bath at 37° C. and PPO (5.0 g) is added in, then xylene is added in to 1 L. Scintillation solution prepared should be kept away from light.

The ConA solution is prepared by the following procedure: 10 mg accurately weighed ConA is dissolved with RPMI 1640 medium sufficiently, make the volume to 100 mL with the concentration of 100 μg/mL.

The LPS solution is prepared by the following procedure: 10 mg accurately weighed LPS is dissolved with RPMI 1640 medium sufficiently, make the volume to 100 mL with the concentration of 100 μg/mL.

Compared with the MTT assay method, the ³H-TdR method has higher sensitivity, better stability and more affordable. For the ³H-TdR method, based on DNA, RNA synthesis in the cell cycle, ³H-TdR can be absorbed into the cells as starting material. Measurement of the intracellular radioactivity of ³H-TdR could reflect cell proliferation.

Spleen lymphocytes include T lymphocytes and B lymphocytes with basically similar content of both. As T lymphocyte mitogen, ConA only promotes the proliferation of T lymphocytes and do not work on the B lymphocytes. On the contrary, LPS can only induce proliferation of B lymphocytes. Crude exopolysaccharides can significantly promote (P<0.01) (Table 6) the proliferation of LPS-activated B lymphocyte, and has a significant dose-dependent manner. Crude exopolysaccharides does not promote the proliferation of in vitro ConA-activated T lymphocyte on mice.

TABLE 6
Effects on T/B lymphocyte proliferation of the crude exopolysaccharides

| | | T lymphocyte | | B lymphocyte | |
|---|---|---|---|---|---|
| | | | percentage of | | percentage of |
| | concentration | | promotion | | promotion |
| | μg/mL | CMP value | (%) | CMP value | (%) |
| negative | — | 332 ± 35 | — | 277 ± 59 | — |
| control | | | | | |
| positive control | — | 24911 ± 822 | — | 11984 ± 1287 | — |
| crude | 10 | 25400 ± 1097 | 2 | 13911 ± 932 | 16 |
| polysaccharides | 100 | 30003 ± 838 | 20 | 20848 ± 1302 | 74 |
| | 1000 | 33131 ± 3648 | 33 | 25066 ± 2608 | 109 |

In vitro lymphocytes culturing experiment shows that exopolysaccharides produced by Lactobacillus brevis BDLB0001 has no cytotoxicity. In vitro immune activity experiment shows that exopolysaccharides produced by Lactobacillus brevis BDLB0001 can significantly promote the proliferation of B lymphocytes and demonstrate a strong immune-enhancing activity.

### Applied Embodiment 1

### Bulk Starter Culture of Lactobacillus brevis BDLB0001

Lactobacillus brevis strain BDLB0001 is inoculated into sterilized 12% (w/w) skimmed milk which is sterilized at 115° C. for 15 min, and cultured at 37° C. for 14-16 h till the milk begin to curd, and the activated culture is continued for two generations, and the products obtained is used as a mother starter culture. Said mother culture is inoculated into new sterilized 12% (w/v) skimmed milk with a ratio of 3-5% (v/v) to culture at the temperature of 37° C. for 14-16 h till the new skimmed milk begin to curd, then said bulk starter culture (1) is obtained with viable bacteria count of about 10⁹ cfu/mL in the curdled milk.

Lactobacillus brevis strain BDLB0001 is inoculated into MRS liquid medium and cultured for activation at the temperature of 37° C. for 14-16 h, and the activated culture is continued for two generations, then the activated culture obtained is inoculated into new MRS liquid medium with a ratio of 2-4% (v/v) to culture for 16-18 h, and then the cell sediment is obtained via centrifugation at the temperature of 4° C. for 15 min with the speed of 4000 r/min, and the cell sediment is suspended in a certain amount of sterilized skimmed milk, then said bulk starter culture (2) is obtained.

### Applied Embodiment 2

### Lactobacillus brevis BDLB0001-Containing Beverage

Raw milk is cooled to 40° C. after pasteurization at the temperature of 95° C. for 20 min or high temperature sterilization at the temperature of 140° C. for 2 s, and then said bulk starter culture (1) or (2) of Lactobacillus brevis BDLB0001 in applied Embodiment 1 is added in to make the concentration of Lactobacillus brevis BDLB0001 up to 10⁶ cfu/mL or more, and then stored at 4° C., i.e., said Lactobacillus brevis BDLB0001-containing beverage is obtained.

### Applied Embodiment 3

### Lactobacillus brevis BDLB0001-Containing Fermented Milk

Raw milk is sterilized at the temperature of 95° C. for 20 min and cooled to 37° C., and then a ratio of 3-5% (v/v) of bulk starter culture (1) or (2) of Lactobacillus brevis BDLB0001 in applied Embodiment 1 as well as mutualistic commercial starter cultures, i.e., Lactobacillus Bulgaria is inoculated into the sterilized milk, and then after being mixed uniformly, the inoculated milk is fermented at 37° C. till the titration acidity reaches to 0.6 calculated by lactic acid, and then stored at 4° C., i.e., said Lactobacillus brevis BDLB0001-containing fermented milk is obtained.

It should be understood that, after reading the contents of the present invention described above, the person skilled in the art can make various modifications of the invention, these equivalent forms also fall within the scope defined by the appended claims of the present application.

1. A strain of exopolysaccharide-secreting Lactobacillus brevis, wherein it is deposited in China General Microbiological Culture Collection Center with an accession number of CGMCC No. 5223.

2. A bulk starter culture of said strain of Lactobacillus brevis according to claim 1, wherein it is prepared according to a method comprising the following step (a) or (b):
(a) inoculating said strain of Lactobacillus brevis according to claim 1 into sterilized milk and culturing till the milk begin to curd, continuing the activated culture for two generations to obtain the mother culture; inoculating said mother starter culture with a ratio of 3-5% (v/v) into new sterilized milk and culturing till the new sterilized milk begins to curd, and then obtaining said bulk starter culture;
(b) inoculating said strain of Lactobacillus brevis according to claim 1 into liquid medium and culturing for activation, continuing the activated culture for two generations, inoculating the activated culture obtained with a ratio of 2-4% (v/v) into new liquid medium and culturing for 16-18 h, obtaining the cell sediment through solid-liquid separation, and suspending the cell sediment in sterilized milk, and then obtaining said bulk starter culture.

3. The bulk starter culture according to claim 2, wherein a viable count of bacteria in said bulk starter culture is no less than 10⁹ cfu/mL.

4. A method of preparing fermented foods, comprising fermenting the food with a bulk starter culture of a strain of Lactobacillus brevis deposited in China General Microbiological Culture Collection Center with an accession number of CGMCC No. 5223.

5. The method according to claim 4, wherein said fermented foods refer to Lactic acid bacteria-containing milk beverage or fermented milk.

6. The method according to claim 5, wherein said Lactic acid bacteria-containing milk beverage is prepared according to a method comprising the following steps: cooling raw milk after sterilization, and mixing with said bulk starter culture of Lactobacillus brevis uniformly to make the concentration of Lactobacillus brevis CGMCC No. 5223 up to 10⁶ cfu/mL or more, and then obtaining said Lactic acid bacteria-containing milk beverage,
further wherein the bulk starter culture is prepared according to a process comprising the following step (a) or (b):
(a) inoculating said strain of Lactobacillus brevis into sterilized milk and culturing till the milk begin to curd, continuing the activated culture for two generations to obtain the mother culture; inoculating said mother starter culture with a ratio of 3-5% (v/v) into new sterilized milk and culturing till the new sterilized milk begins to curd, and then obtaining said bulk starter culture;
(b) inoculating said strain of Lactobacillus brevis into liquid medium and culturing for activation, continuing the activated culture for two generations, inoculating the activated culture obtained with a ratio of 2-4% (v/v) into new liquid medium and culturing for 16-18 h, obtaining the cell sediment through solid-liquid separation, and suspending the cell sediment in sterilized milk, and then obtaining said bulk starter culture.

7. The method according to claim 5, wherein said fermented milk is prepared according to a method comprising the following steps: cooling raw milk after sterilization, inoculating a ratio of 3-5% (v/v) of bulk starter culture of Lactobacillus brevis as well as a ratio of 3-5% (v/v) of mutualistic commercial starter cultures into the sterilized milk, mixing uniformly and fermenting till the titration acidity reaches to 0.6-0.7 calculated by lactic acid, and then obtaining the fermented milk containing said Lactobacillus brevis,
further wherein the bulk starter culture is prepared according to a process comprising the following step (a) or (b):
(a) inoculating said strain of Lactobacillus brevis into sterilized milk and culturing till the milk begin to curd, continuing the activated culture for two generations to obtain the mother culture; inoculating said mother starter culture with a ratio of 3-5% (v/v) into new sterilized milk and culturing till the new sterilized milk begins to curd, and then obtaining said bulk starter culture;
(b) inoculating said strain of Lactobacillus brevis into liquid medium and culturing for activation, continuing the activated culture for two generations, inoculating the activated culture obtained with a ratio of 2-4% (v/v) into new liquid medium and culturing for 16-18 h, obtaining the cell sediment through solid-liquid separation, and suspending the cell sediment in sterilized milk, and then obtaining said bulk starter culture.

8. Exopolysaccharides extracted from said strain of Lactobacillus brevis according to claim 1.

9. The exopolysaccharides according to claim 8, wherein a method of extraction comprises the following steps: centrifuging the fermentation liquor of said strain of Lactobacillus brevis according to claim 1 after boiling in order to remove thalli and condensed protein, precipitating protein from supernatant via trichloroacetic acid method to remove the protein, obtaining a precipitate by adding alcohol, dissolving the precipitate in water and then dialyzing with water.

10. A method of enhancing immunity of a subject in need thereof, comprising administering to the subject with an immune-enhancing medicament, a healthcare product or a kind of food product comprising an effective amount of the exopolysaccharides according to claim 8.

11. The method according to claim 10, wherein the method of extraction comprises the following steps: centrifuging the fermentation liquor of said strain of Lactobacillus brevis according to claim 1 after boiling in order to remove thalli and condensed protein, precipitating protein from supernatant via trichloroacetic acid method to remove the protein, obtaining a precipitate by adding alcohol, dissolving the precipitate in water and then dialyzing with water.


SEQUENCE LISTING (1 sequence)

SEQ ID NO: 1 - DNA, 1442 bp, Lactobacillus brevis
cttagacggctgactcccgaaggttatctcaccggctttgggtgttacaaactctcatggtgtgacgggcggtgtgtacaaggcccgggaacgtattcaccgcggcatgctgatccgcgattactagcgattccaacttcatgtaggcgagttgcagcctacaatccgaactgagaacggctttaagagattagcttagcctcacgacttcgcgactcgttgtaccgtccattgtagcacgtgtgtagcccaggtcataaggggcatgatgatttgacgtcatccccaccttcctccggtttgtcaccggcagtctcaccagagtgcccaactgaatgctggcaactgataataagggttgcgctcgttgcgggacttaacccaacatctcacgacacgagctgacgacaaccatgcaccacctgtcattctgtccccgaagggaacgtcttatctctaagattggcagaagatgtcaagacctggtaaggttcttcgcgtagcttcgaattaaaccacatgctccaccgcttgtgcgggcccccgtcaattcctttgagtttcaaccttgcggtcgtactccccaggcggagtgcttaatgcgttagctgcagcactgaagggcggaaaccctccaacacttagcactcatcgtttacggcatggactaccagggtatctaatcctgttcgctacccatgctttcgagcctcagcgtcagttacagactagacagccgccttcgccactggtgttcttccatatatctacgcattccaccgctacacatggagttccactgtcctcttctgcactcaagtctctcagtttccgatgcacttctccggttaagccgaaggctttcacatcagacttaaaaaaccgcctgcgctcgctttacgcccaataaatccggacaacgcttgccacctacgtattaccgcggctgctggcacgtagttagccgtggctttctggttaaataccgtcaacccttgaacagttactctcaaaggtgttcttctttaacaacagagttttacgagccgaaacccttcttcactcacgcggcattgctccatcagactttcgtccattgtggaagattccctactgctgcctcccgtaggagtttgggccgtgtctcagtcccaatgtggccgattaccctctcaggtcggctacgtatcatcgtcttggtgggcctttacctcaccaactaactaatacgccgcgggatcatccagaagtgatagccgaagccacctttcaaacaaaatccatgcggattttgttgttatacggtattagcacctgtttccaagtgttatcccctgcttctgggcagattccccacgtgttactcaccagttcgccactcgcttcattgttgaaatcagtgcaagcacgtcattcaacggaagctcgttcgacttgcatgt